(19) United States
(12) Patent Application Publication
(10) Pub. No.: US 2021/0228103 A1
(43) Pub. Date: Jul. 29, 2021

(54) SYSTEM AND ELECTRONIC CIRCUIT FOR CONDITIONAL INFORMATION TRANSFER AND METHOD THEREFOR

(71) Applicants: Wolfgang Richter, Vancouver (CA); Faranak Zadeh, Vancouver (CA)

(72) Inventors: Wolfgang Richter, Vancouver (CA); Faranak Zadeh, Vancouver (CA)

(73) Assignee: R2Z INNOVATIONS, INC., VANCOUVER (CA)

(21) Appl. No.: 14/687,939

(22) Filed: Apr. 16, 2015

(51) Int. Cl. A61B 5/0537; A61B 5/00; G06K 7/10

(52) U.S. Cl. CPC A61B 5/0537; H02M 7/02; G06K 7/10366; A61B 5/4872

(57) ABSTRACT

An electronic system for triggering and transporting conditional information from a user or an item, the system comprising at least one each of electronic circuit, carrier, rectifier, buffer, source, level shifter, system clock, sync unit, communication unit, interpreter and/or bidirectional interface; a set of electrodes for recognizing 3D-gestures of a user and at least one bidirectional integrated display, wherein sync unit synchronizes the electronic circuit with the source by briefly (e.g. for 1 millisecond) interrupt alternating electric charges and/or sound waves and/or vibrations, and/or also reacts to changes caused by external modulation and bringing logic elements into a defined state and separates the clock from data for facilitating introduction of information, data or commands into the electronic circuit and interpreter interprets received information or data into the electronic circuit as commands and forwards them over level shifter to the interface to operate the integrated (e.g. e-ink, LCD or rheological) display, which is also used as touch sensor.

### TECHNICAL FIELD AND INDUSTRIAL APPLICABILITY OF THE INVENTION

The present invention relates to an electronic chip or an electronic circuit which can harvest energy and powers itself to work as a sensor and a communicator without requiring any battery. The present invention further relates to a system based on the electronic chip or circuit configured for conditional information transfer. The present invention also relates to a method for conditional information transfer by means of the system and electronic circuit therefor.

### CROSS-REFERENCE TO RELATED PATENTS

Not Applicable.

### BACKGROUND OF THE INVENTION

U.S. Pat. No. 8,611,828 (U.S. Ser. No. 13/174,613) discloses a new kind of biological-sensing controller, based on silicon and/or flexible polymer printed electronics. The purpose of the device is to monitor and/or control biological signals of living organisms (for example, microbes, bacteria, insects, plants, animals, and people). Embedded in a system, the innovation can work contactless and battery-free, since it is self-powered, wirelessly self-communicating without using electromagnetic waves like radio frequencies (RF), infrared or other electromagnetic technologies. In contrast, the innovation uses alternating electric fields for powering, measuring and communicating, and introduces an innovative new method of mobile vital signs monitoring. Accordingly, a flexible sensing action (“SenseAction”) technology can be used for example in a shop/retail store, whereby the price of a product may be changed, when a customer is identified to be given an incentive to shop. For this purpose, a harvester, a communicator, a processor and a switch matrix are integrated on a foil and attached to an e-ink display in a shelf. A hub sends the information of the product and the customer to the internet, where such “little data” becomes “big data” for analysing purposes. Further, as shopping and paying are related actions, the flexible tags can be attached to money-clips or banking cards to identify customers when they go for shopping or perform financial activities. However, there is still a need for a system and an electronic circuit for conditional information transfer and a method therefor.

### BRIEF DESCRIPTION OF THE INVENTION

Conditional information can be generated or transferred based on the location, movements or action of a user or items. Information can be triggered by an event caused by a user with a special interest or behaviour. The art provides systems and methods based on input devices, such as buttons or sensors, computing and/or processing devices, such as MCUs, FPGAs, SOCs, etc. as well as feedback devices for information, such as speakers, screens, signalling, illumination, digital signage, LCD, etc. However, if a plurality of such devices is needed, then a variety of challenges are present:
1. Cost: Combining various discrete parts, ICs, printed circuit boards, enclosures, power supplies, wires, networks; investments in design, production, assembly, marketing and maintenance make such systems too expensive for many applications.
2. Focus: A user can trigger information using a smart device, such as a phone or a tablet during a specific action, for example—while shopping (or driving). However, this shifts the focus of a user from a product (or situation) to the operating needs of said smart device, which can distract or alter the purpose of the situation. Focusing on triggering the information not only turns the attention of a user, but also changes the priority of a situation and creates extra, mostly unwanted action.
3. Power: Sensing devices and other electronic circuits need energy to operate, which is normally stored in DC-buffers, such as batteries or accumulators, also known as button cells. Such elements need either frequent recharging, if possible, or replacement and recycling, which creates additional efforts and/or proves to be a burden for the environment. Sometimes it is not allowed to put toxic chemicals from such elements in combination with other products, for example—food, livestock, or within the reach of small children. In fact, providing efficient power to mostly wireless remote sensing systems is one of the greatest challenges of the information technology in this decade.
4. Microwaves: Wireless sensing requires wireless connectivity normally used with electromagnetic means, such as radio-waves or infrared radiations. Microwave systems, such as Bluetooth or Wi-Fi are very popular, but their concentration at one single location can present enormous challenges. Concentrated microwaves can be harmful for humans, pets or plants. Microwaves can kill microorganisms. Radio-waves can cause interference, reflections, absorptions or shadows caused by objects in the reach of their beams.
5. Safety: As microwaves spread in sphere, they are easy to monitor and wireless networks are often misused or attacked by hackers or criminals. Triggering conditional information is often is a very private process between users, users and items, or situations of their interest. Privacy is a big concern these days. Information systems are often used to analyse a user's behaviour, which makes it easier to manipulate him/her, create need, as well as turn-over for the manipulators. The so-called “big-data”, is a good example that industry plans to precede to manipulate billions of prospective consumers.

The invention related solutions provide a system and an electronic circuit to trigger or transport conditional information from a user or an item in a specific situation. The invention puts the user in the center of the action, at a short distance (e.g. in reach) from “self-powered sensing and communication devices”, which cannot be monitored from a certain distance. The system is invisible and keeps the user's focus on the main action and the purpose of the situation without any further or unwanted distraction.

The invention uses printable or flexible electronics for the sake of cost and simple installation. The electronic circuit contains means to harvest, store or transport electrical energy without additional external parts.

The invention can also be in any shape, like a symbol (for example as the logo of a brand) and can be attached to products, shelves, packages or other items and combines the functions of powering, sensing and communication in a single electronic circuit.

### BRIEF SUMMARY OF THE INVENTION

In accordance with the teachings of the present invention, there is provided an electronic system for conditional information transfer, which triggers and transports conditional information from a user or an item in a particular situation, the system comprises:
(a) at least one electronic circuit within reach of or attached on a flexible carrier, the circuit includes at least one conductive surface for coupling alternating electric charges and/or sound waves and/or vibrations;
(b) at least one rectifier to convert the alternating electric charges and/or sound waves and/or vibrations into a direct current and voltage as captive energy to be used for powering the electronic circuit;
(c) at least one buffer for storing direct current (DC);
(d) at least one source emitting alternating electric charges and/or sound waves and/or vibrations;
(e) at least one level shifter for receiving the alternating electric charges and/or sound waves and/or vibrations from a source and to shift these changes to a different level;
(f) and transferring the same into a system clock subcircuit;
(g) at least one system clock receiving the alternating electric charges and/or energy caused by sound waves and/or vibrations from the level shifter;
(h) at least one sync unit to synchronize the electronic circuit with the source for avoiding data collision;
(i) at least one communication unit sub-circuit for outgoing and incoming communication over the source;
(j) at least one interpreter;
(k) at least one bidirectional interface;
(l) at least one set of electrodes for recognizing 3D-gestures, or changes in impedance e.g. of a user; and
(m) at least one bidirectional integrated display or feedback unit;

wherein the sync unit synchronizes the electronic circuit with the source, if the latter is briefly (e.g. for 1 millisecond) interrupting the alternating electric charges and/or sound waves and/or vibrations, and/or also reacts to changes therein caused by external modulation and thus brings logic elements into a defined state and to separate clock from data for facilitating introduction of information, data or commands into the electronic circuit.

Some of the objects of the present invention—satisfied by at least one embodiment of the present invention—are as follows:

An object of the present invention is to provide an electronic circuit for conditional information transfer.

Another object of the present invention is to provide a method for conditional information transfer for sending data from the electronic circuit to a source or external data receiver.

Still another object of the present invention is to provide an electronic circuit powered without a battery and having sub-circuits and interface for recognizing the 3D gestures of a user for contactless data transfer and communication.

Yet another object of the present invention is to provide an electronic circuit for sensing approaching user or changes in impedances and communicating the information related to the product of user's interest.

Still further object of the present invention is to provide an electronic circuit for interpreting the user's interest and interpreting his/her gestures to launch the information on a feedback device.

Yet further object of the present invention is to provide an electronic circuit with an internal gesture recognition system for enhancing or replacing the touch functions of a display.

Still another object of the present invention is to provide a method for contactless measurement of changes in impedance over time and user information.

Yet another object of the present invention is to provide a method for conditional information transfer by means of the system and electronic circuit therefor.

### BRIEF DESCRIPTION OF DRAWINGS

The present invention will be briefly described with reference to the accompanying drawings, wherein:

FIG. 1 shows the system for conditional information transfer in accordance with the present invention.

FIG. 2 shows the sub-circuit for generating the internal clock, sync and receiving data used in the system shown in FIG. 1.

FIG. 3 shows the principal underlying the present invention for transfer data between the innovative electronic circuit and a source or external data receiver.

FIG. 4 shows an exemplary combination of a mechanical generated and an alternating charges energy wave for operating an electronic circuit in accordance with the present invention, which is attached on the handle of a shopping card.

FIG. 5 shows the sub-circuit for recognizing the 3D gestures in accordance with the present invention.

FIG. 6 shows the invention moulded into a car's tire's rubber for detecting obstacles, environmental changes or getting external local information.

### DETAILED DESCRIPTION OF DRAWINGS

In the following, the present invention will be described in more details with reference to the accompanying drawings without limiting the scope and ambit thereof in any way.

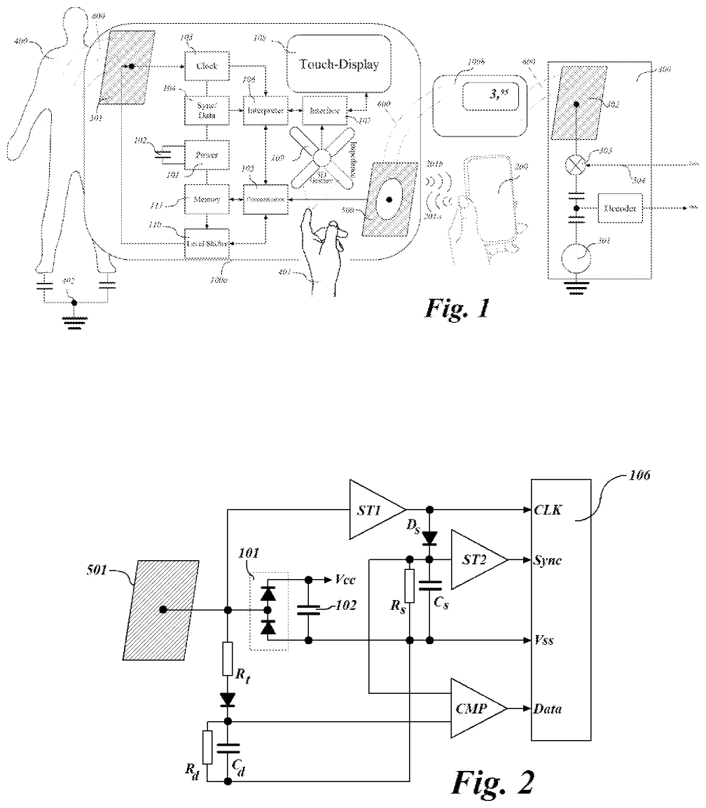

FIG. 1 shows an electronic circuit attached on a flexible carrier, 100a or 100b. The circuit contains a conductive surface (electrode 501) to couple alternating electric charges with users or items and/or ground. [Here, a combi-element 500 is also provided, which is a combination of piezo element with its surface used as another conductive coupling surface (electrode). However, in some other products or embodiments, the combi-element 500 may not be necessary. The combi-element 500 can couple with alternating electric charges as well as with sound-waves of specific frequency (for example, resonance frequency). The combi-element 500 can also emit sound waves or alternating electric charges (even at the same time). Sound waves or mechanical vibrations cause the combi-element 500 to generate voltage and electrical energy, which can also be used by the electronic circuit of the invention.] Alternating electric charges, sound waves or vibrations received from the electrode 501 and/or combi-element 500 will be rectified by the power unit 101 and stored in a buffer (e.g. capacitor) 102 which can be a printed rechargeable battery, a silicon battery, a gold cap or any other battery or energy storage device, preferably flat in dimensions. Alternating electric changes (from external sources) received from electrode 501 and/or electric charges and/or vibrations from combi-element 500 can be transferred and triggered into a system clock 103 from the level shifter 110. Since such “waves” are emitted either from an external smart device (200) or an external source (300), the frequency which is triggered and transferred into the level shifter 110 will be the same of any invention related electronic circuit within reach (for example 100a and 100b). Therefore, these circuits are synchronized with the source's frequency, which is an important fact to avoid collision in data communication. A sync unit 104 can synchronize the circuit and bring its logic elements into a defined state. To synchronize the system and electronic circuit in accordance with the present invention, the external source briefly interrupts the alternating electric charge or sound waves/vibrations, for example—for about 1 millisecond. The sync unit 104 also reacts to the alterations in the electric charges, which can be caused by external modulation. This makes it possible to bring information/data or commands into the circuit, which will be described later in detail. Once received, data can be interpreted as commands from an interpreter, which is a kind of hardware, decoder or state-machine 106. Depending on the command, interpreter 106 can either send data or information to a communication unit 105 which emits it over a level shifter 110 over 501 and/or 500; or interpreter 106 sends data to an interface 107 which works bidirectional. Interface 107 can operate an integrated display 108 (for example, e-ink, LCD, rheological, etc.), which can also be used as a touch sensor. Therefore, the display 108 works bidirectional. Electric fields 600 are charges, which can be easily absorbed, bridged or altered by other surfaces, for example—the user's hand 401. In a special arrangement, electrodes from a subcircuit 109 can be used to recognize 3D gestures (e.g. in 1-2 ft. range), which will be transported over the interface 107 to the interpreter 106 which interprets the gestures and informs external devices 300 over the communication unit 105, the level shifter 110, the electrode 501 and/or combi-element 500 (also an electrode). Another special aspect of the invention is the obstacle avoidance capability of the 3D gesture system with electrodes 109, which will be described later. The (preferable) flexible carrier 100a can be within reach or attached to a smart device 200 (for example, attached to a smart phone as a sticker on the back side or on its accessories). Therefore, if the smart device 200 generates, for example—vibrations, the Piezo-Combi element 500 can separate energy, clock and data from the acoustic or mechanical waves to power and operate the circuit on flexible carrier 100a. Sensing results can be returned to the smart device 200 via the piezo combi-element 500 in the form of (ultra-) sound waves or mechanical vibrations, which can be detected from the microphone or MEMs of the smart device 200. Vibrations or sound waves 201a (if generated by smart device 200) or 201b (if generated by combi-element 500) can be of various frequencies (preferably resonance frequency) in combination of audible (can be heard), ultra- or subsonic (e.g. vibration) frequency. An electronic circuit in an external source or enclosure 300 can act as a power source and a hub for data or information at the same time. A generator 301 can generate a periodical signal of a certain level, which emits alternating electrical charges on the electrode 302, which is a conductive surface. Other surfaces within reach, for example—the ones in 100b mirror such charges, also known as capacitive coupling. The mirrored charges then can be rectified and stored and used as electrical energy to operate the circuit in 100b. The charge can also be bridged to 100a; received by combi-element 500 (for example) and grounded capacitive coupled over electrode 501 and a user 400 to ground 402 to close the circuit. As this is realized like an AC circuit, all components inside the circuit work as impedances, so any changes can be detected by the electrodes 109, interpreted by interpreter 106, communicated by communication unit 105 over the combi-element 500 and/or electrode 501, if necessary, shifted to a different level by the level shifter 110. The normal coupling means disposed between 300, 100b, 100a or 400 is air. But it is also possible to put items in between which work as dielectric. A dielectric is also an impedance by which changes can be detected and communicated. The electronic circuit in external source 300 also has a mixer 303 to mix data into the electric field 600 emitted by electrode 302. Every unit (e.g. 100a or 100b) of the invention has a clock unit 103 and a sync unit 104, which can also separate data from the clock and lead it to the interpreter 106. Therefore, the commands from an external source (200 and/or 300) can be given over 303 into devices within reach. As said earlier, the electronic circuit in flexible carrier 100a or 100b can also communicate over the piezo combi-element 500 with acoustic/mechanical waves or by modulating the alternating electric fields 600 in different variations (ASK, FSK, spreadsheet, spread spectrum, pulse shift, phase modulation, etc.) and such modulations can be coupled out to the decoder 304 and can be sent as data into the networks for triggering, feedback or analysing, computing or processing purposes.

FIG. 2 shows the sub-circuit for generating the internal clock, sync and for receiving data. The data-decoder sub-circuit shown in FIG. 2 is responsible for system clock synchronization and data receiving. After electric charges appear on the electrode 501 (either alternating e-field or converter sound or vibrations), sub-circuit 101 rectifies the charges and converts them into DC energy, which is stored on the buffer 102 (to power the connected sub-circuits). A typical buffer can be a capacitor, a gold cap, a rechargeable battery, a printed battery, a silicon battery, etc. However, the person skilled in the art may conceive many other possible embodiments within the scope of the technology described herein. The analog periods of the sine waves need to be digitized to work as a clock, which is done by Schmitt-trigger ST1 having a threshold and hysteresis. The clock on its output is then forked to the sync sub-circuit as well as to all other sub-circuits requiring a clock. The output of ST1 provides the clock, for example, to the interpreter 106 or other clock-operated sub-circuits of the electronic circuit of the invention. The sync sub-circuit consists of another Schmitt-trigger ST2 which has its input connected to a sync capacitor Cs which is connected in parallel to a resistor R_{S}. Over a rectifier (e.g. ESD) or synchronized switch, the capacitor C_{S} is charged with the clock signal above the Schmitt-trigger threshold, so ST2 triggers. Once the clock source disables the clock to provide a sync signal, the sync capacitor C_{S} discharges over the resistor R_{S} below the threshold level and the Schmitt-trigger ST1 stops triggering, which can be interpreted as sync signal to reset or synchronize the internal circuitry. As long as the clock is present, positive pulses will be directed over diode D_{S} into the capacitor C_{S} which is discharged by R_{S}. C_{S} charges over the threshold level from Schmitt-trigger ST2, but discharges over R_{S}, for example with an interruption of 1 millisecond. If no pulse signal is coming from ST1, it means that the external source (300 or 200) has stopped generating the waves. In this case, the output of ST1 stays permanently negative and C_{S} discharges over R_{S}, so that ST2 also becomes negative (logic 0) which can be represented as sync signal from the system. If the clock returns, the output of ST2 becomes positive (logic 1). The interpreter 106 contains an analog/digital switch matrix, which is routed in such a way that data received and held in, e.g. a shift register can be hardware interpreted, for example, to switch on/switch off segments on the display or routing gesture measurement results or memory information to the communication unit. All invention related devices within reach of an alternating electric field or a sound source receive the same clock, therefore the related sync units synchronize all of them at the same time. All signals and voltage levels are referenced against (V_{cc}), which is directly connected to the buffer 102. For receiving data from the external sources (200, 300), the invention provides another sub-circuit with a comparator (CMP), which gets a reference from C_{S}, with modulated data arriving over the electrode 501. The sine wave is directed over Rₜ and a diode to the capacitor C_{d} and builds a charge over the threshold level of the opposite input of the comparator CMP. C_{d} is much smaller than C_{S}, so it discharges quicker over R_{d}. This happens, if the waves contain alterations in their amplitude or frequency (or phase), while ST1 still generates the clock from the exactly the same wave. If such a value is below the reference value, the comparator CMP switches on and this appear as a logical signal on the interpreter 106, which can be shifted into a shift register under the control of the clock. This also means that the data transfer is synchronized with the source. The interpreter 106 can interpret the data as a command in order to set internal switching or routing, for example—to the display.

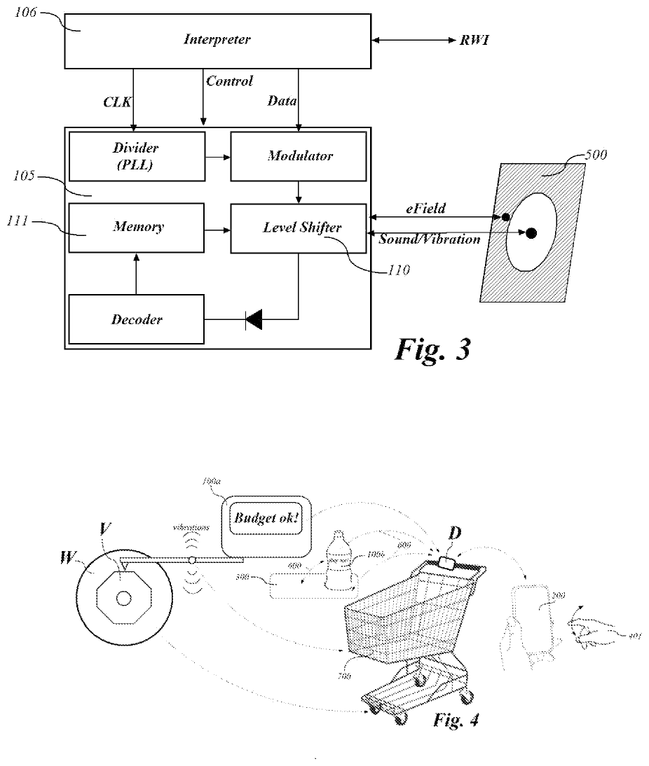

FIG. 3 shows the principal underlying the present invention for sending data from the electronic circuit of the invention to an external source or external data receiver. The clock comes to a divider or multiplier to generate a sub-carrier for the data transfer. Data from the invention related electronic circuit is routed by the interpreter, the gesture system, the memory or other sub-circuits into a modulator, which modulates the electric field charges as well as the mechanical waves for sound and vibration for the piezo combi-element 500. If combi-element 500 is within reach or in contact with a smart device, the mechanical or audible waves can be received from the internal microphone or MEMs circuit of the smart device. An app in a smart device can control the functions related to the received data. The speaker of the smart device can generate pulses which can be received from piezo combi-element 500 and the invention's sub-circuits demodulate them as data and power themselves with the pulses' energy at the same time, as already described. The invention allows combining of data transfer over alternating electrical charges and generated mechanical waves. Whenever such waves appear, the energy will be used for conversion into a DC voltage, stored into the invention related electronic circuit's buffer and can be used to operate the sub-circuits. Data (ID number, codes, measurements values, memory, etc.) is transmitted on demand from or to the external sources 200 or 300, which is handled by interpreter 106. The synchron clock is sent from 106 to 105, which builds a sub-circuit for outgoing and incoming communication over the piezo/e-field combi-element 500. The clock is normally in LF range (e.g. 125 kHz) and it needs to be divided to generate vibrations or sonic pulses on the piezo part of combi-element 500. Since the clock is a digital signal, the divider can be realized as a chain of flip-flops. It may be necessary for some applications that carrier frequencies have to be generated which are higher than the synchronous frequency, therefore the invention provides a phase locked loop, PLL, which can generate any required carrier frequency. The data from interpreter 106 is mixed with the carrier in a modulator, for example using pulse code modulation PCM. For example, a 4 KB carrier can send out Manchester coded pulses to be compatible with RFID systems. Sub-circuit 110 is a level shifter and a fork, which brings the electric field at a predetermined voltage level and directs it to the e-field emitting part of the piezo-electrode or combi-element 500. If the frequencies are lower in sounds and vibrations, sub-circuit 110 directs the modulated data to the sound vibration part (piezo of the Combi-element 500). Signals received by combi-element 500 are forked out and separated from a carrier and decoded and stored in the memory 111 under the control of interpreter 106. The data decoder is already described in FIG. 2.

FIG. 4, shows an example of a mechanically generated energy wave for operating a flexible carrier 100a, which is attached on the handle of a vehicle, such as a shopping cart (or basket) 700 normally used in a retail space. At least one wheel W of the shopping cart 700 is attached with a mechanism V (e.g. an uneven surface), which generates vibrations. The invention related flexible carrier 100a is attached with a sub-circuit and a display D is mounted on the handle of the shopping cart 700 and is influenced by the vibration, when the shopping cart 700 is moving. Whenever such shopping cart 700 comes close to another electric field 600, for example from the external source 300 placed in the shelves, on which a product, for example a bottled product 800 such as milk with a label 100b is stored, it can get energy and thus can share data and communicate with it. Products 800 can be attached with the electronic circuits of the invention in the form of stickers (so-called SmartLogos). If a consumer puts any such product 800 in a shopping cart 700, it will be registered by the flexible carrier 100a and can be transferred to external sources 300 or 200. A consumer can carry a smart phone equipped with a flexible carrier 100a in the form of a sticker (digital tattoo, SmartLogo, etc.) on the backside of the smart phone. The smart phone carried by the consumer can energize this system equipped with the electronic circuit of the invention and thus can send and receive data over the human body to and from the shopping cart 700, products 800 or external sources 200, 300 within reach. Since the flexible carrier or sticker 100a attached to the smart phone is also integrated with a gesture recognition sub-circuit, a consumer can make 3D gestures within reach of his/her smart phone, which can be interpreted as commands, for example—to store data or to provide information. The vibrations can be received by the combi-element 500 (piezo electrode) in accordance with the invention, which uses the metallic construction of the shopping cart, for example, as electrode 501 or as an extension. There can be a number of external sources 300 mounted along a shopping area of a retail store, which all emit electric fields 600 and data. If the shopping cart 700 comes within reach of such electric fields 600, it mirrors the charges to create DC voltage in accordance with the invention. This means that the presence of a shopping cart 700 at a certain location can be detected, if the attached external source 100a sends out an ID code. A consumer could also carry a unit 100a attached to a smart phone 200 on or near his/her body which contains a user ID. The invention related waves spread out over the consumer's body and the shopping cart 700 and can give the user ID to said external sources 300 within reach. One location can be the cash register: if the consumer comes close with his/her ID, s/he will be recognized and served as a frequent buyer. As products 800 can also be equipped with the invention's electronic circuit in the form of smart logos or smart labels, their identification codes can be combined with the consumer's ID and the location ID presented from the system connected to external sources 300. Especially when a user touches a product 800, this brings the closest possible energy transfer, which is detectable and transferable as a trigger signal for information related to the product or service from the location or brand.

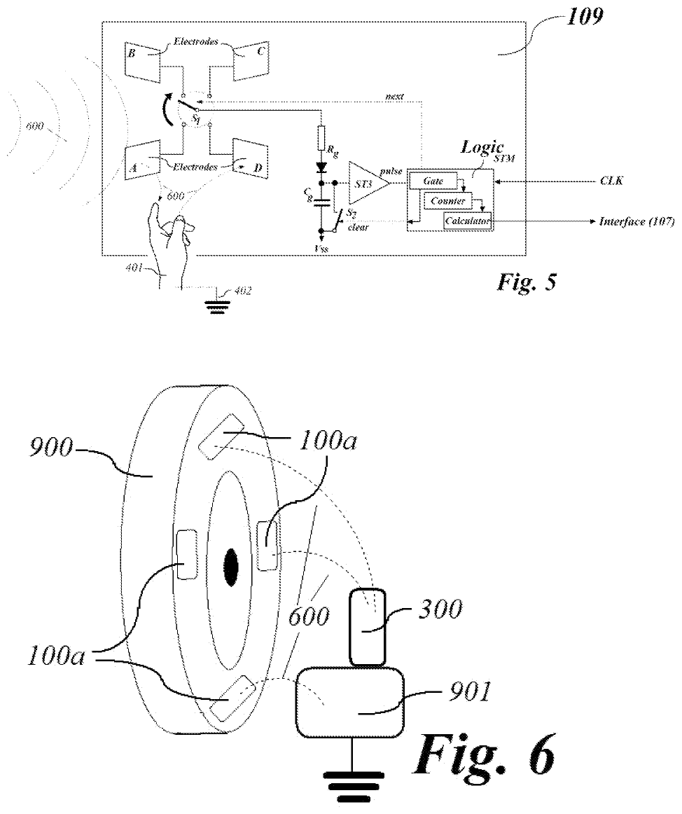

FIG. 5 describes the gesture sub-circuit 109 in detail. A logic sub-circuit is clocked by the synchronous system clock and switches an electronic switch S₁ periodically between the sub-electrodes A, B, C, D which are embedded, for example—in the corners of the flexible carriers 100a. As the source frequency is preferably stable, it not only provides a clock for the system and the counter, but also acts as a reference for the analog to digital conversion of the sensing part of the invention. The electrodes A, B, C, D receive the alternating electric field and rectify it over a resistor R_{g} and the capacitor C_{g}. After a predetermined threshold, Schmitt-trigger ST3 generates a pulse which forces a logic gate to start the counter, counting by the Clock frequency and also discharging the capacitor C_{g} over an electronic switch S₂. Depending on the absorption or bridging of the human hand 401, the next threshold for the Schmitt trigger ST3 will be reached in a different time from one electrode than from the others of the sub-electrodes A, B, C, D, which causes different results in the counter. A calculator calculates the difference of the previous counter results for every sub-electrode A, B, C, D and sends these results to the interface 107, which is connected to the interpreter 106. The calculator then sets the result as a previous result and makes it as the base for the next calculation. This provides that the system does not stuck on obstacles and works even if a user holds the invention attached smart device in his hands or near his body or covered with clothes. Those skilled in the art may conceive many other possible variations or embodiment within the scope of the technology described herein.

The invention can be used in many applications; some of them are listed below. The smart device (Smart Phone, Smart Watch, tablet, wearable computing, etc.) also can contain circuitry to generate alterinating e-field and modulate or demodulate charges.

### Applications

### Contactless 3D Gesture Interface

The electronic circuit of the invention is preferably attached to a flexible carrier (e.g. sticker), which can be shaped and printed as a brand's logo or as a kind of electronic tattoo. Such sticker can be attached on the backside of a smart device (smart phone, tablet, smart watch, etc.). A special app can generate sub-sonic or ultrasonic vibrations to power the tattoo, which emits an electric field 600 and activates the integrated gesture sub-circuit to measure and interpreted changes in the field, for example caused by a human action with limbs or body. Typical reach around a smart device is 20 to 60 cm (2 feet). The gestures and their values are interpreted and sent back to the smart device in form of vibration or sound signals which can be received either from a microphone or the integrated MEMs circuit and computed or processed in smart devices application or sent to networks to provide or trigger information.

Electronic shelf labels: The invention is attached to a shop's shelf, which is influenced from a unit 300, e.g. by an alternating electric field 600. The labels have displays to show a price of a related product 800. If a consumer approaches, he/she brings an approaching ground potential to such label over the conductivity and surface of his/her body. The approach can be sensed and communicated to the unit 300, which prepares information related to the product 800 of interest. If the consumer touches the product 800 his gestures are also interpreted and can launch the information on a feedback device, for example, using digital screens (TFT screens or projectors or other augmented display technologies). A consumer can have or carry the invention as a kind of:

Credit card or customer card: such card is activated whenever an alternating electric field is detected and special commands are received over the field. Such card can contain an internal energy buffer 102 as described earlier, which can be recharged for example from a vibration source, which can also be the human body itself during sport and fitness and other activities which cause vibration or mechanical pulses (shocks). As the integrated electronic of the invention is realized in ultra-low power technology, the energy of the internal buffer last 100s of data transactions without being recharged, but can recharged in a short time, for example placing sources 300 as a kind of HUB everywhere in the space that the user is moving. If he/she comes close to such a HUB, energy is transported over the human body to the invention related buffer and stores energy as direct current (DC) for use of measurements or changing information (for example, identification codes (ID)).

Switching functions: The buffer contains more energy than the internal sub-circuits need to operate, therefore it can be provided to power external devices. For example, illumination like LED, OLED, electro florescent, laser, etc. especially in different colors can make products more attractive and more informative for a consumer. For example giving warning of allergies or signals of monitored ingredients or situations.

The interface 107 also can have external I/O (real world interface, RWI) to switch external devices ON or OFF under the control of the interpreter 106, and commands from devices 200 or 300. In a special application for a car or other vehicle such invention can stop “texting while driving” which can lead to terrible accidents. If a driver on a car seat is influenced by an electric field, all devices in his hands can be disabled from specific functions, while passengers don't have these restrictions because there is no e-field presence over their body. The invention's electronic circuit can be combined with smart devices to switch internal functions on or off.

The internal gesture recognition system of the invention can also be used to enhance or replace the touch functions of a display. Furthermore, gestures around a device, which is equipped with the invention, can be also recognized; even gestures from several users or several limbs at once (3D multi-gesture). Also, movements can be sensed and interpreted for speed and direction, which can be a special feature to detect and analyze movements of persons in reach of 100b (e.g. as shelf labels).

On a flexible carrier 100a, the invention can be attached to handles of bicycles, shopping carts, steering wheels, pedals, medical instruments, musical instruments, remote controls, smart devices of any kind, or attached on plants or animals. The electrodes 500 or 501 can be extended with conductive material (polymers, metals, wires, thread etc.).

Human can carry the invention as patches or electronic tattoos direct on the body, or on clothes, shoes, gloves, belts, hats, bags, apparel, etc.

Smart Rubber: A very special application of the invention is to mould the electronic circuit into a more flexible carrier, e.g. rubber. As rubber contains carbon, it can be used as a carrier and an e-field (e.g. 500) electrode (or as an extension for an electrode) at the same time, which means that the rubber product can be a very versatile (wireless) sensor. The invention can be moulded into tires, seals, mats, tapes, etc. (in some cost sensitive application, the piezo part of the invention may not be necessary). The SmartRubber can be used as a touch or gesture pad or to measure impedances or their charges.

Printed electronics: The invention's electronic circuit is also designed to be realized in printable electronic, hybrids or combination of printable electronic and silicon. It is also possible to use a piezo element (501) as a carrier for a chip with the invention related circuit integrated. This leads to applications like buttons, jewelleries (e.g. rings, necklace, wristbands), accessories for smart devices, nails or pins, metal batches, plastic batches, symbols, printed stickers and labels of any material, picks for instruments, etc.

Remote impedance measurement: The frequency of a generated alternating electric field can be spanned over an interval (for example from 10 kHz to 500 kHz) and emitted over said electrode 302. Instead of gestures, the sensing system 109 can react on changes in the impedance of the object in between the electrodes.

As impedance can change over time, e.g. in implants, living organisms, or food products 800, the electronic circuit in accordance with the present invention provides a contactless measurement of such changes, which can trigger related action or user information. For example, a human can be qualified on his body mass, fat and bones (also known as BMI).

Molded into a car's tires 900, the electronic circuit according to the present invention can announce a “too close” approach to pavement stones 901 or other obstacles which may cause damage to car parts. The tire also can receive data from 300 units in reach (e.g. parking meters) or environmental changes, e.g. street condition. The inventive electronic circuit or sources 300 can also be attached to smart devices in the form of (replaceable) modules (like phone blocks, connector plugs etc.). Those skilled in the art will envision many other possible variations within the scope of the technology described herein.

Technical Advantages of the Invention-related Electronic Circuit: The electronic circuit is accordance with the present invention can be configured as I-Dots, which can be used as under:
(a) In smart devices—
   (i) as phone blocks (ARA),
   (ii) as integrated and add-on features in smart phones, and
   (iii) as clip-on features in smart phones;
(b) As hubs as predetermined locations, e.g. shelves; and
(c) At home—
   (I) On power outlet,
   (II) For smart TV,
   (III) In remote control, and
   (IV) In electronic keys.

These I-Dots can have diverse applications exemplarily mentioned below:
(A) In retail space:
   As electronic labels,
   In intelligent (sensing) shopping carts,
   For customer identification,
   In electronic packaging,
   Smart products labels,
   SmartLogos,
   For sales support, and
   On smart shelves.
(B) In smart devices, such as:
   In smart cradles, e.g. gesture-controlled cradles and housing,
   For Signalling,
   For audio-processing, e.g. in smart microphones, earbuds and audio implants,
   As personal “Internet of Things” (IoT) hubs.
(C) In markets:
   In textile products, e.g. smart buttons, shoes, gloves and headbands,
   For fashion industry, as smart watches, jewelry, glasses and unique textile effects,
(D) In consumer electronics:
   In home appliances, white goods (refrigerators, washing machines etc.) as well as in brown goods (cooking stoves, cookware),
   For gesture control in music,
   In remote controls for gesture, signalling and identification purposes,
   In connected homes for smart metering and signalling, in smart furnitures, for monitoring presence, child protection and smart alert/protection services,
   In smart carpets and
   For aging at home;
(E) In brands:
   For smart packaging,
   SmartLogos,
   Sales support, and
   Peel'n use;
(F) For generic uses to intelligently sense:
   Touch, Gesture, Pinch,
   Signalling RGB,
   Audio, and
   Physical forces.

The main advantages of I-Dots are enumerated as under:
1. Require no additional external parts,
2. Facilitate contactless sensing,
3. Powers peripherals,
4. Enable RF-free wireless communication,
5. Configurable as printable electronics,
6. Flat and flexible,
7. Require no soldering,
8. Use no toxic chemicals and
9. Very cost effective, costing just about 1 Cent/Chip.

Throughout this specification the word “comprise”, or variations such as “comprises” or “comprising”, shall be understood to implies including a described element, integer or method step, or group of elements, integers or method steps, however, does not imply excluding any other element, integer or step, or group of elements, integers or method steps.

The use of the expression “a”, “at least” or “at least one” shall imply using one or more elements or ingredients or quantities, as used in the embodiment of the disclosure in order to achieve one or more of the intended objects or results of the present invention.

The exemplary embodiments described in this specification are intended merely to provide an understanding of various manners in which this embodiment may be used and to further enable the skilled person in the relevant art to practice this invention. The description provided herein is purely by way of example and illustration.

Although the embodiments presented in this disclosure have been described in terms of its preferred embodiments, the skilled person in the art would readily recognize that these embodiments can be applied with modifications possible within the spirit and scope of the present invention as described in this specification by making innumerable changes, variations, modifications, alterations and/or integrations in terms of materials and method used to configure, manufacture and assemble various constituents, components, subassemblies and assemblies, in terms of their size, shapes, orientations and interrelationships without departing from the scope and spirit of the present invention.

While considerable emphasis has been placed on the specific features of the preferred embodiment described here, it will be appreciated that many additional features can be added and that many changes can be made in the preferred embodiments without departing from the principles of the invention. These and other changes in the preferred embodiment of the invention will be apparent to those skilled in the art from the disclosure herein, whereby it is to be distinctly understood that the foregoing descriptive matter is to be interpreted merely as illustrative of the invention and not as a limitation.

What is claimed is:

1. An electronic system for triggering and transporting conditional information from a user or an item in a particular situation, the system comprises:
(a) at least one electronic circuit within reach of or attached on a flexible carrier, the circuit includes at least one conductive surface for coupling alternating electric charges and/or sound waves and/or vibrations;
(b) at least one rectifier to convert the alternating electric charges and/or sound waves and/or vibrations into a direct current and voltage as captive energy to be used for powering the electronic circuit;
(c) at least one buffer for storing direct current (DC);
(d) at least one source emitting alternating electric charges and/or sound waves and/or vibrations;
(e) at least one level shifter for receiving the alternating electric charges and/or sound waves and/or vibrations from the source and to shift these changes to a different level and transferring the same into a system clock;
(f) at least one system clock unit receiving the alternating electric charges and/or sound waves and/or vibrations from the level shifter;
(g) at least one sync unit to synchronize the electronic circuit with the source;
(h) at least one communication unit for building a sub-circuit for outgoing and incoming communication over the source;
(i) at least one interpreter;
(j) at least one bidirectional interface;
(k) at least one set of electrodes for recognizing 3D-gestures, or changes in impedance, e.g. of a user; and
(l) at least one bidirectional integrated display or feedback unit;
wherein the sync unit synchronizes the electronic circuit with the source by briefly (e.g. for 1 millisecond) interrupting the alternating electric charges and/or sound waves and/or vibrations, and/or reacts to changes therein caused by external modulation and brings logic elements into a defined state and separates clock from data for facilitating introduction of information, data or commands into the electronic circuit.

2. Electronic circuit as set forth in claim 1, wherein the interpreter is a kind of hardware decoder or state machine interpreting the information or data received by the electronic circuit as commands and forwards the same over the level shifter to the interface to operate the integrated display, such as e-ink, LCD or rheological display also used as a touch sensor.

3. Electronic circuit as set forth in claim 1, wherein the source is an external source, comprising a piezo combi-element, separating electric energy, clock and data from the acoustic or mechanical waves to power the electronic circuit on a flexible carrier and the circuit includes at least one memory to store ID or decoded signals received by the piezo combi-element, which are forked out and separated from the carrier and the interpreter controls the memory over the communication unit.

4. Electronic circuit as set forth in claim 3, wherein the source is a smart device, e.g. a smart phone to provide energy, clock and data via the piezo combi-element in form of (ultra-)sound waves or mechanical vibrations to be detected by the microphone or MEMS of the smart device.

5. Electronic circuit as set forth in claim 3, wherein the external source includes another electronic circuit functioning as a power source and a hub for information and data at the same time and a first flexible carrier within reach mirrors alternating electric charges as capacitive coupling, the mirrored charges are rectified, stored and used as electrical energy for operating the electronic circuit in the first flexible carrier and the mirrored charges are bridged to a second flexible carrier to be subsequently received by piezo combi-element and capacitive coupled over the electrode and the user to be finally grounded to the ground for closing the circuit.

6. Electronic source circuit as set forth in claim 1, wherein the electronic circuit comprises:
at least one generator to generate a periodical signal of a predetermined level to emit an alternating electric charge on at least one conductive surface or electrode;
at least conductive surface or electrode;
at least one mixer to mix data into the electric field emitted by electrode; and
at least one decoder for coupling modulations of the alternating electric fields in different variations (ASK, FSK, spreadsheet, spread spectrum, pulse shift, phase modulation, etc.)
means to communicate information with smart devices or networks;
wherein commands from the external source are given over the mixer to smart device/s within reach and the electronic circuit in the flexible carrier communicates over the piezo combi-element with acoustic/mechanical waves or by modulations of the alternating electric fields and the decoder sends these modulations as data into the networks for triggering, feedback or analysing, computing or processing purposes.

7. Electronic circuit as set forth in claim 6, wherein the electronic circuit of the decoder comprises:
a rectifier sub-circuit for converting alternating electric charges or acoustic waves or vibrations into direct current (DC) energy;
a buffer for storing this DC energy;
a first Schmitt trigger for converting analog input signals, e.g. sine waves into digital output signal to function as clock;
a synchronization sub-circuit including a second Schmitt trigger; and
an interpreter circuit coupled at the output of the second Schmitt trigger;
wherein the first Schmitt trigger digitizes analog signals to work as clock signal; a sync capacitor is connected in parallel to a resistor at the input of the second Schmitt trigger; the second Schmitt trigger triggers on charging the sync capacitor with the clock signals having a value above the first Schmitt trigger threshold and the output of the second Schmitt trigger becomes positive (logic 1) and the second Schmitt trigger stops triggering on discharging the sync capacitor with a value below the first Schmitt trigger threshold, e.g. with an interruption of 1 milliseconds; when no pulse signal is forwarded by the first Schmitt trigger, the output of the first Schmitt trigger becomes permanently negative to make the output of the second Schmitt trigger negative (logic 0), represented as sync signal from the system.

8. Electronic circuit as set forth in claim 7, wherein interpreter circuit interprets the received data as commands and accordingly either sends data or information to a communication unit, to be emitted over a level shifter over the electronic circuit; or interpreter sends data to a bidirectional interface operating an integrated display, which can also be used as a touch sensor.

9. System as set forth in claim 1, wherein the electronic circuit is configured as an AC circuit and components inside the electronic circuit work as impedances for detecting any changes in the alternating electric charges by means of electrodes.

10. System as set forth in claim 1, wherein the coupling means disposed between the external source, first and second flexible carriers and user is air or a dielectric, which detects the changes in the impedance and communicates the same over the electrode.

11. System as set forth in claim 1, wherein the vibrations or sound waves generated by smart device or vibrations or sound waves generated by piezo combi-element consist of various frequencies (preferably resonance frequency) in combination with audible (can be heard), ultra- or subsonic (e.g. vibration) frequency.

12. System as set forth in claim 1, wherein the system comprises:
a first flexible carrier attached on a vehicle (e.g. shopping cart) with at least one wheel W thereof fitted with a vibration generating mechanism for generating vibrations during movement thereof and the handle thereof fitted with a display;
on coming closer to an electric field from at least one external source placed in a location, the vehicle receives energy from the external source for sharing data and for communicating therewith;
a second flexible carrier configured with a predetermined sub-circuit is placed on a product or an item, for example—in the form of stickers, the item being placed in a place (for e.g. shelves) or other locations; and
a smart phone of a user equipped with a third flexible carrier energizes the system for sending and receiving data over user to and from vehicle, items or external source;
wherein the vehicle generates vibrations during movement thereof, which are received by the piezo combi-element, vehicle (e.g. shopping cart) and external source placed along the shopping emit electric field and data, which are mirrored by the vehicle to create a direct current (DC) voltage detected at a predetermined location when an external source send out an ID code or when a customer carrying a flexible carrier attached to a smart device on or near his/her body is recognized by means of alternating electric charges or sound waves or vibration emanating from the vehicle spread out over his/her body by giving his/her ID to at least one other external source located within reach.

13. System as set forth in claim 12, wherein the products equipped with the flexible carrier and identification of the product combined with the customer ID and location ID provided by the system connected to the external source, triggers signal or information related to the product or service by the customer touching the product.

14. System as set forth in claim 1, wherein the electronic circuit attached to the labels on a shop's shelf, responds to the alternating electric field of an external source placed within reach, the labels include an electronic display for showing the price of a related product stored on the shelf, wherein customer approaching the shelf is sensed by approaching ground potential towards such label over the conductivity or body surface of the customer and communicated to the external source to prepare information related to the product of customer's interest or the gestures of the customer on touching the product or the label are interpreted to launch relevant information on at least one feedback device, such as digital screen, (TFT screens or projectors or similar augmented display technologies) included in the labels for encouraging or tempting him/her for special action.

15. System as set forth in claim 14, wherein the electronic circuit with an internal buffer is embedded in a card (e.g. customer card, royalty card, credit card, membership card, passes, or ticket) for activation by means of an alternating electric field detected in the vicinity of the customer carrying the credit card, to receive special commands over the field and to be recharged from a source of alternating e-field or ultrasound waves, or as vibrations or mechanical pulses during physical activities, the recharge lasting for a number of transactions; and/or wherein the internal buffer is recharged in a short time from an external source placed as a hub around the space in which the customer is moving, by transporting energy over customer's body to the buffer, to store it as direct current (DC) for transferring the conditional information or for communicating using ultra-low power technology.

16. System as set forth in claim 12, wherein the internal buffer contains excess energy than is needed for operating the plurality of sub-circuits in the electronic circuit, for providing the excess energy to power external devices, such as LED, OLED, electro-fluorescence, Laser etc. for making items more visible or for providing information, such as warnings or signals of preferred ingredients or other information to a user or networks.

17. System as set forth in claim 1, wherein the interface includes an external I/O or Real World Interface (RWI) to switch on/off the smart device under the control of the interpreter and by receiving commands from the smart device or external source, such as texting during driving, the smart device adapts to be switched off when the driver is sitting on his/her seat fitted with the electronic circuit and the electric field adapts to be activated to disable all devices in driver's hands from predetermined functions, while the passengers do not have any such restrictions due to absence of such e-field over their body.

18. System as set forth in claim 1, wherein the electronic circuit moulded into a rubber product containing an electrically conductive material like carbon, for example tire of a motor vehicle, whereby the rubber product configured as a carrier and as an e-field electrode functions as a sensor to avoid obstacles, such as pavement stones or informational systems equipped with a plurality of external sources, by transferring data and communicating the alternating electric charges or sound waves or vibrations present in the vicinity of the electronic circuit or by measuring changes in the impedance thereof, in order to announce a “too close” approach to pavement stones or other obstacles or conditions.

19. System as set forth in claim 1, wherein the electronic circuit embedded in wearables like buttons, jewelleries, accessories for smart devices, nails or pins, plastic batches, symbols, plectrums, printed stickers or labels of any flexible material etc. is produced as a printable circuit or chip incorporating the piezo combi-element as a carrier thereof.

20. System as set forth in claim 1, wherein the electronic circuit includes a sensing electrode to react on sensing the changes in impedance of the item in between such electrodes, for example—to ascertain Body Mass Index (BMI) of a human body or for remote measurement of the changes in environment, implants, living organisms or food products combined with information.
